(19) United States
(12) Patent Application Publication
(10) Pub. No.: US 2025/0295732 A1
(43) Pub. Date: Sep. 25, 2025

(54) COMPOSITIONS AND METHODS FOR ACTIVATING INNATE IMMUNITY

(71) Applicant: Zoetis Services LLC, Parsippany, NJ (US)

(72) Inventors: Brianna Joy Pomeroy Beam, Kalamazoo, MI (US); Hilary Phelps, Jackson, MI (US); Adriann Marie Mootz, Kalamazoo, MI (US); Xi Zeng, Kalamazoo, MI (US)

(21) Appl. No.: 18/719,464

(22) PCT Filed: Aug. 5, 2022
(86) PCT No.: PCT/US2022/074569

§ 371 Date: Jun. 13, 2024

Related U.S. Application Data
(60) Provisional application No. 63/289,709, filed on Dec. 15, 2021.

(51) Int. Cl. A61K 38/17; A61P 31/04; A61P 37/04

(52) U.S. Cl. CPC A61K 38/1729; A61P 31/04; A61P 37/04

(57) ABSTRACT

The invention relates to compositions and methods for activating innate immunity. Specifically, the invention relates to cathelicidin peptides for activating or inducing innate immunity.

### CROSS-REFERENCE TO RELATED APPLICATIONS

This application claims priority to and the benefit of U.S. Provisional Patent Application 63/289,709, filed Dec. 15, 2021, which is incorporated by reference herein in its entirety.

### FIELD OF THE INVENTION

The invention relates to compositions and methods for activating innate immunity. Specifically, the invention relates to cathelicidin peptides for activating or inducing innate immunity.

### BACKGROUND OF THE INVENTION

Bovine animals are exposed to numerous potential pathogens daily, through contact, ingestion, and inhalation. The ability to avoid infection depends in part on the adaptive immune system, which remembers previous encounters with specific pathogens and destroys them when they attack again. However, the adaptive immune responses are slow to develop on first exposure to a new pathogen, as specific clones of B and T cells have to become activated and expand. It can therefore take at least a week before the responses are effective. By contrast, a single bacterium with a doubling time of one hour can produce millions of progenies, a full-blown infection, in a single day. Therefore, during the first critical hours and days of exposure to a new pathogen, the bovines rely on their innate immune system to protect them from infection.

Innate immune responses are not specific to a particular pathogen in the way that the adaptive immune responses are. They depend on a group of proteins, phagocytic cells (e.g., monocytes, macrophages, neutrophils, dendritic cells, and mast cells), and non-phagocytic cells (e.g., NK cells) that recognize conserved features of pathogens and become quickly activated to help destroy invaders. Therefore, the quick activation of innate immune system is critical for animals.

Some bovine animals have the problem of an inability to induce or activate their innate immune memory. This could be due to a mechanistic disorder in a group of proteins and phagocytic cells that recognize conserved features of pathogens. These animals have an inactivated innate immune memory. They are in need of an activated or induced innate immune memory in order to provide an immunity protection against a pathogen, a disease or a condition.

Accordingly, there exists a need for compositions and methods for activating or inducing innate immune memory in bovine.

### SUMMARY OF THE INVENTION

In one aspect, the invention relates to a method for activating or inducing innate immune memory in a bovine in need thereof, the method comprising: administering to said bovine an effective amount of cathelicidin 2 (CATH2) peptide or a variant thereof, thereby activating or inducing innate immune memory in said bovine.

In another aspect, the invention relates to a method for treating a condition of an inactivated innate immune memory in a bovine subject, the method comprising: administering to said bovine an effective amount of cathelicidin 2 (CATH2) peptide or a variant thereof, thereby treating said condition of the inactivated innate immune memory in said subject.

In yet another aspect, the invention relates to a composition comprising: cathelicidin 2 (CATH2) peptide or a variant thereof, wherein said peptide or its variant is present in said composition in an amount effective to activate or induce innate immune memory in a bovine. In an exemplary embodiment, the composition is an intra-mammary delivery composition.

In a further aspect, the invention relates to a delivery device comprising: a chamber for storing a composition, wherein said composition comprises cathelicidin 2 (CATH2) peptide or a variant thereof, wherein said peptide or its variant is present in said composition in an amount effective to activate or induce innate immune memory in a bovine. In an exemplary embodiment, the device is a mammary delivery device.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that CATH2 trained bovine monocyte-derived macrophage have enhanced LPS-induced cytokine response. Cytokine levels were measured in supernatants from bovine monocyte-derived macrophages stimulated with LPS (1 μg/mL) for 24 hours following exposure to vehicle control or CATH2 for 24 hours, removal of CATH2 and vehicle, and a 3-day rest period. Cytokines were quantified using U-Plex MesoScale Discovery internally developed assay. Mean with SD.

FIG. 2 shows CATH2-trained bovine monocyte-derived macrophage LPS-induced lactate response. Lactate levels were measured in supernatants from bovine monocyte-derived macrophages stimulated with LPS (1p g/mL) for 24 hours following exposure to vehicle control or CATH2 for 24 hours, removal of CATH2 and vehicle, and a 3-day rest period. Lactate was quantified using LactateGlo® Promega assay. Mean with SD.

FIG. 3 shows histone modification changes in CATH2-trained bovine monocyte-derived macrophage pre- and post-LPS stimulation. Monocyte-derived macrophages exposed to vehicle control or CATh2 for 24 hr then rested for 3 days. After 3-day rest, a subset of cells was harvested for histone isolation prior to LPS stimulation (A) and a second subset were stimulated with LPS (1 μg/mL) for 24 hr and then histone modifications were isolated (B). H3 modifications were measured via ELISA using Epigentek kits. Fold change represents fold change of CATH2-treated cells over vehicle control cells for each histone modification as a % total H3. Mean with SD.

FIG. 4 shows ChIP-Seq peak calling analysis of CATH2-trained bovine monocyte-derived macrophage pre-LPS stimulation compared to vehicle control monocyte-derived macrophages. Monocyte-derived macrophages exposed to vehicle control or CATH2 for 24 hr then rested for 3 days. After 3-day rest, a subset of cells was harvested for chromatin isolation prior to LPS stimulation and chromatin samples were submitted to Diagenode for ChIP-Sequencing for H3K27ac histone modification. Principal component analysis plot (A) and venn diagram of shared and unique peaks across samples for H3K27ac mark (B).

FIG. 5 shows CATH2-trained bovine monocyte-derived macrophage LPS-induced phenotypic surface marker expression. Surface marker expression was measured on bovine monocyte-derived macrophages stimulated with LPS (1 μg/mL) for 24 hours following exposure to vehicle control or CATH2 for 24 hours, removal of CATH2 and vehicle, and a 3-day rest period. Mean with SD.

FIG. 6 shows the expression of activation markers in the milk macrophage population (defined as CD172+CD14+ cells) and the effect of CATH2 treatment on surface marker expression 12 hr following E. coli intramammary challenge. CD14, CD80, MHC II, and CD163 surface marker expression increased in CATH2-treated quarters 12 hours post-challenge suggesting CATH2 reprogrammed the local macrophage response to E. coli challenge. T01=4×100 mg of CATH2/10 mL intramammary dose at 12 hr intervals starting 48 hr prior to challenge (0 hr, 12 hr, 24 hr, and 36 hr prior to challenge), T02=1×400 mg of CATH2/10 mL intramammary dose 48 hr prior to challenge, T03=4×10 mL vehicle control intramammary dose at 12 hr intervals starting 48 hr prior to challenge, T04=1×10 mL vehicle control intramammary dose 48 hr prior to challenge. Graphs depict fold change of CD14+CD172a+ milk cells geometric mean fluorescence intensity for each surface marker in CATH2-treated quarter over respective CD14+CD172a+ milk cells geometric mean fluorescence intensity for each surface marker in vehicle control quarter for each animal; each animal had a CATH2-treated quarter and a vehicle control quarter for within cow comparison. Data are graphed as individual animal with median+/−interquartile range.

FIG. 7 shows the comparison of gene module (ME1, ME2, ME5, ME8, ME9, ME11) expression pattern across time between CATH2 and vehicle control group in bovine monocyte-derived macrophages over time. 0 hrs=Prior to CATH2 treatment, 8 hrs=8 hours following exposure to CATH2 or vehicle, 24 hrs=24 hours following exposure to CATH2 or vehicle, 0 hrsLPS=Prior to LPS challenge and following CATH2 exposure and 3-day rest period, 8 hrsLPS=8 hours post LPS challenge and following CATH2 exposure and 3-day rest period, 24 hrsLPS=24 hours post LPS challenge and following CATH2 exposure and 3-day rest period.

FIG. 8 shows of heatmap of cytokines expression log 2 (Fold change) between CATH2 and vehicle control group contrast across time points in bovine monocyte-derived macrophages. 8 hrs=8 hours following exposure to CATH2 or vehicle, 24 hrs=24 hours following exposure to CATH2 or vehicle, 0 hrsLPS=Prior to LPS challenge and following CATH2 exposure and 3-day rest period, 8 hrsLPS=8 hours post LPS challenge and following CATH2 exposure and 3-day rest period, 24 hrsLPS=24 hours post LPS challenge and following CATH2 exposure and 3-day rest period.

FIG. 9 shows the expression of macrophage associated activation markers in bovine monocyte-derived macrophages and the effect of CATH2 treatment on surface marker expression 8 hr following LPS stimulation. CD14 and CD80 surface marker expression significantly increased in CATH2-treated monocyte-derived macrophages 8 hours post-LPS suggesting CATH2 reprogrammed monocyte-derived macrophages by modulating gene expression of these activation markers.

FIG. 10 shows CATH2-trained bovine monocyte-derived macrophage enhances phagocytosis of E. coli bioparticle. Lactate levels were measured in supernatants from bovine monocyte-derived macrophages stimulated with E. coli bioparticle (10 μg/mL) for 24 hours following exposure to vehicle control or CATH2 for 24 hours, removal of CATH2 and vehicle, and a 3-day rest period. Green fluorescence and live cell image analysis was captured on the IncuCyte® S3 (Essen Bioscience). Geometric mean with 95% confidence intervals.

FIG. 11 shows CATH2 trained bovine monocyte-derived macrophage have enhanced E. coli bioparticle-induced reactive nitrogen species (RNS) response. Nitrite levels were measured in supernatants from bovine monocyte-derived macrophages stimulated with E. coli bioparticles (10 μg/mL) for 24 hours following exposure to vehicle control or CATH2 for 24 hours, removal of CATH2 and vehicle, and a 3-day rest period. Nitrite were quantified using Griess Reagent System (Promega). Mean with SD.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

### Definitions

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (animal or human) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be, for example, an inactivated innate immune memory.

The terms “subject,” “individual,” and “patient” are used interchangeably herein, and refers to an animal to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), bovine, sheep, goat, dog, cat, rodent, (e.g. mouse or rat), guinea pig, pig, rabbits, horses and non-mammals such as reptiles, amphibians, chickens, and turkeys.

### CATH2 Molecules

The invention provides CATH2 peptide and variants thereof. The inventors of the instant application have surprisingly and unexpectedly found that the bovine innate immune memory can be effectively activated by the administration of CATH2 peptide or its variants.

The term “peptide” as used herein, refers to a sequence of amino acids coupled by a peptide bond, wherein the amino acids are one of the twenty naturally peptide-building amino acids and wherein one or all of the amino acids can be in the L-configuration or in the D-configuration, or, for isoleucine and threonine in the D-allo configuration (only inversion at one of the chiral centers). A peptide according to the invention can be linear, i.e. wherein the first and last amino acids of the sequence have a free NH₂— or COOH-group respectively or are N-terminally (acetylation) and/or C-terminally (amidation) modified.

As used herein, the terms “CATH2” and “CMAP27” are used interchangeably. Like other members of the cathelicidin family CMAP27 is encoded as a prepropeptide (154 amino acids) and after proteolytic processing, a C-terminal peptide is released that has demonstrated potent broad-spectrum antimicrobial activity.

The amino acid sequence of this C-terminal peptide, called CMAP27 or CATH2, is RFGRFLRKIRRFRPKVTITIQGSARFG (SEQ ID NO.: 1) or its truncated functional sequence RFGRFLRKIRRFRPKVTITIQ (SEQ ID NO.: 35). The term, “CMAP27” or “CATH2,” as used herein, refers to either the 27 amino acid sequence set forth in SEQ ID NO.: 1 or the 21 amino acid sequence set forth in SEQ ID NO.: 35.

As used herein, a “CATH2 derivative” generally refers to a peptide that is a derivative of CATH2 in that it contains at least part of the sequence of CATH2 and that has maintained at least one antimicrobial properties of CATH2, although not necessarily to the same extent. In particular, antimicrobial activity against Gram(−) bacteria, Gram(+) bacteria, or a combination thereof is maintained.

As used herein, the term “variant” may refer to a structural or functional variant including, for example, analogs or derivatives of CATH2 peptide.

In one embodiment, the CATH2 derivative is selected from the group consisting of C-terminally and/or N-terminally truncated CATH2 derivatives, D-amino acid CATH2 derivatives, C-terminally or N-terminally truncated D-amino acid CATH2 derivatives, cyclic CATH2 derivatives and inverso and retroinverso CATH2-derivatives. The derivative or analog may contain one or more amino acid substitutions, preferably 1 to 5 amino acid substitutions, more preferably 1, 2, 3 or 4 amino acid substitutions. Preferably, the CATH2 derivative is selected from the group consisting of C-terminally and/or N-terminally truncated CATH2 derivatives, D-amino acid CATH2 derivatives and C-terminally or N-terminally truncated D-amino acid CATH2 derivatives, such as C-terminally or N-terminally truncated DCATH2. In one preferred embodiment, CATH2 or DCATH2 is used. DCATH2 may include the full length CATH2 peptide having D-amino acids.

“C-terminally truncated CATH2 derivatives” refers to truncated peptides lacking one or more amino acids at the C-terminus of CATH2, preferably lacking up to 17 amino acids, more preferably up to 12 amino acids, more preferably up to 6 amino acids. The examples are described in WO 2010/093245, which is incorporated herein by reference, and especially the peptides listed as CMAP26-NH₂, CMAP26, CMAP26 (P14→G), CMAP26 (P14→L), CMAP1-21, CMAP1-15, CMAP1-15 (F2→L), CMAP1-15 (F5→L), CMAP1-15 (F12→L), CMAP1-15 (3xF→L), CMAP1-15 (F2→W), CMAP1-15 (F5→W), CMAP1-15 (F12→W), CMAP1-15 (F2→W; F5→W; F12→W), CMAP1-13, CMAP1-12, CMAP1-11 and CMAP1-10 in Table 1 of said document and their acetylated and/or amidated derivatives. Herein, and in all amino acid sequence defined herein, the arrow notation indicates an amino acid substitution. For instance, F2→L indicates that the F at position 2 is replaced by L and F2, 5→W indicates that F at positions 2 and 5 is replaced by W. Further preferred are CMAP1-21 (F2→W), CMAP1-21 (F5→W), CMAP1-21 (F2→W), CMAP1-21 (F2→W), CMAP1-21 (F5, 12→W), CMAP1-21 (F2, 12→W), CMAP1-21 (F2, 5, 12→W), CMAP1-21 (F2→Y), CMAP1-21 (F5→Y), CMAP1-21 (F12→Y), CMAP1-21 (F2, 5→Y), CMAP1-21 (F5, 12→Y), CMAP1-21 (F2, 12→Y), CMAP1-21 (F2, 5, 12→Y), CMAP1-21 (F2→W; F5→Y), CMAP1-21 (F2→Y; F5→W), CMAP1-21 (F5→W; F12→Y), CMAP1-21 (F5→Y; F12→W), CMAP1-21 (F2→W; F12→Y), CMAP1-21 (F2→Y; F12→W), CMAP1-21 (F2→W; F5→Y; F12→Y), CMAP1-21 (F2→Y; F5→W; F12→Y), and CMAP1-21 (F2→Y; F12→Y; F12→W).

The examples of C-terminally truncated CATH2 derivatives are also described in WO2015/170984, which is incorporated herein by reference. The CMAP proteins identified above, may also be indicated as CATH2 peptides. CMAP1-21 then would be CATH2(1-21).

“N-terminally truncated CATH2 derivatives” are CATH2 derivatives that are truncated at the N-terminal amino acid (arginine) of CATH2 thus lacking one or more amino acids at the N-terminus of CATH2, preferably lacking up to 10 amino acids, more preferably up to 7 amino acids, more preferably up to 6 amino acids. Examples of the N-terminally truncated CATH2 derivatives include, but not limited to, N-terminally truncated variants of CMAP1-21: CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y).

“D-amino acid CATH2 derivatives” are CATH2 derivatives as defined herein (including the above defined C- and N-terminally truncated CMAP27-derivatives) that contain at least one amino acid in the D configuration. A special category of these D-amino acid CATH2 derivatives are the peptides that are composed of only D amino acids (i.e. in which no L amino acid is present). This special category is herein defined as DCATH2. Also CATH2 itself, comprising one or more, or, alternatively, all D amino acids is comprised within this definition. In one embodiment, D-amino acid CATH2 derivatives are DCATH2. In some embodiments, the invention includes the following examples of D-amino acid CATH2 derivatives (indicated as D-C, and where all amino acids are in the D-form):

| | |
|---|---|
| | (SEQ ID NO.: 17) |
| D-C(1-26) | RFGRFLRKIRRFRPKVTITIQGSARF-NH₂ |
| | (SEQ ID NO.: 18) |
| D-C(1-21) | RFGRFLRKIRRFRPKVTITIQ-NH₂ |
| | (SEQ ID NO.: 19) |
| D-C(4-21) | RFLRKIRRFRPKVTITIQ-NH₂ |
| | (SEQ ID NO.: 20) |
| D-C(7-21) | RKIRRFRPKVTITIQ-NH₂ |
| | (SEQ ID NO.: 21) |
| D-C(7-21)F/W | RKIRRWRPKVTITIQ-NH₂ |
| | (SEQ ID NO.: 22) |
| D-C(7-21)F/Y | RKIRRYRPKVTITIQ-NH₂ |
| | (SEQ ID NO.: 23) |
| D-C(10-21)F/W | RRWRPKVTITIQ-NH₂ |
| | (SEQ ID NO.: 24) |
| D-C(1-15) | RFGRFLRKIRRFRPK-OH |

In a particular embodiment, DCATH2 derivative is DCATH2(1-21) (also called DC(1-21)) or DCATH2(4-21) (also called DC(4-21)).

“Cyclic CATH2-derivatives” are CATH2 derivatives in which at least two non-adjacent amino acids are connected to form a ring structure. Although in principle any chemical binding construction may be used, such as replacing two non-adjacent amino acids in any of the above-mentioned CATH2 derivatives with a cysteine, where these cysteines then form an S-S bridge, a preferred binding system uses the binding between Bpg (Fmoc-L-bishomopropargylglycine) and an azido-resin, wherein the Bpg is attached to an internal arginine, leucine, phenylalanine or tryptophane residue and the azido-resin is attached to the C-terminal glutamic acid residue.

Non-limiting examples such cyclic derivatives are below:

cycCMAP(1-21)[Lys8]
(SEQ ID NO.: 2)
RFGRFLR(Bpg)IRRFRPKVTITIQ(azido-resin)
cycCMAP(1-21)[Arg7]
(SEQ ID NO.: 3)
RFGRFL(Bpg)KIRRFRPKVTITIQ(azido-resin)
cycCMAP(1-21)[Leu6]
(SEQ ID NO.: 4)
RFGRF(Bpg)RKIRRFRPKVTITIQ(azido-resin)
cycCMAP(1-21)[Leu6], Phe2/Trp
(SEQ ID NO.: 5)
RWGRF(Bpg)RKIRRFRPKVTITIQ(azido-resin)
cycCMAP(1-21)[Leu6], Phe2, 5/Trp
(SEQ ID NO.: 6)
RWGRW(Bpg)RKIRRFRPKVTITIQ(azido-resin)
cycCMAP(1-21)[Leu6], Phe2, 5, 12/Trp
(SEQ ID NO.: 7)
RWGRW(Bpg)RKIRRWRPKVTITIQ(azido-resin)
cycCMAP(1-21)[Leu6], Phe5, 12/Trp
(SEQ ID NO.: 8)
RFGRW(Bpg)RKIRRWRPKVTITIQ(azido-resin)
cycCMAP(1-21)[Leu6], Phe12/Trp
(SEQ ID NO.: 9)
RFGRF(Bpg)RKIRRWRPKVTITIQ(azido-resin)

“Inverso” and “Retroinverso” CATH2 derivatives (“I”-CATH2 and “RI”-CATH2 derivatives) are peptides that have an inverted sequence with respect to the above-mentioned CATH2 derivatives, in the sense that the amino acids are connected to each other in a reverse order. When the inverted CATH2 derivatives contain one or more D amino acids they are termed “Retroinverso” or “RI”. If the inverted derivative only contains L-amino acids it is termed “Inverso” or “I”. The I and RI equivalent of CATH2 then may become GFRASGQITITVKPRFRRIKRLFRGFR (SEQ ID NO.: 10). Other non-limiting examples of such I or RI-CMAP27-derivatives are:

| | |
|---|---|
| | (SEQ ID NO.: 11) |
| RI-C(1-21) | QITITVKPRFRRIKRLFRGFR |
| | (SEQ ID NO.: 12) |
| RI-C(4-21) | QITITVKPRFRRIKRLFR |
| | (SEQ ID NO.: 13) |
| RI-C(7-21) | QITITVKPRFRRIKR |
| | (SEQ ID NO.: 14) |
| RI-C(7-21)F/W | QITITVKPRWRRIKR |
| | (SEQ ID NO.: 15) |
| RI-C(7-21)F/Y | QITITVKPRYRRIKR |
| | (SEQ ID NO.: 16) |
| RI-C(10-21)F/W | QITITVKPRWRR |

The I and RI-CMAP27 derivatives may be acetylated at their N-terminal and/or amidated at their C-terminal.

In a particular embodiment, the CATH2 or derivative thereof used in any method or use of the invention is CATH2, DCATH2, DCATH2(1-21), DCATH2(4-21), CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (E5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) or CMAP10-21 (F12→Y). In some embodiments, the CATH2 or derivative thereof used in any method or use of the invention is CATH2, DCATH2, DCATH2(1-21) or DCATH2(4-21). In one embodiment, the CATH2 or derivative thereof used in any method or use of the invention is DCATH2, DCATH2(1-21) or DCATH2(4-21).

In some embodiments, the CATH2 or derivative thereof used in any method or use of the invention is one or more the peptides below.

```
  (SEQ ID NO.: 25)
RCGRFLRKIRPFRRKVTITRQ
(SEQ ID NO.: 26)
RCGRFLRKIRPFRGKVTITRQ
(SEQ ID NO.: 27)
RFGRFLRKIRRFRGKVTITRQ
(SEQ ID NO.: 28)
RWGRWLRKIRRWRPKVTITRQ
  (SEQ ID NO.: 29)
RWGRWLRKIRRWRPKVTITIQ
  (SEQ ID NO.: 30)
RFLRKIRRFRPKVTITRQ
  (SEQ ID NO.: 31)
RFLRKIRRFRGKVTITRQ
  (SEQ ID NO.: 32)
RWLRKIRRWRPKVTITIQ
  (SEQ ID NO.: 33)
RWLRKIRRWRPKVTITRQ
  (SEQ ID NO.: 34)
RWLRKIRRWRGKVTITRQ
(SEQ ID NO.: 35)
RFGRFLRKIRRFRPKVTITIQ
(SEQ ID NO.: 36)
RCGRFLRKIRPFRRKVTITCQ
  (SEQ ID NO.: 37)
RFGRWLRKIRRYRGKVTITIQ
```

In one exemplary embodiment, the peptide of the invention is an immune modulatory peptide having no antibiotic activity because CATH2's intrinsic antimicrobial activity is abrogated with milk or milk proteins. In another exemplary embodiment, the peptide of the invention is an immune modulatory peptide having no direct killing effect on bacteria because CATH2's intrinsic antimicrobial activity is abrogated with milk or milk proteins.

### Methods for Producing Peptides

Methods for producing peptides are well known in the art and fully described in U.S. Patent Application Publication 20170145065, which is incorporated by reference herein in its entirety. Any suitable method can be used for making the peptides of the invention. In one embodiment, the peptides of the invention are produced synthetically. Peptide chemical synthesis techniques are well known in the art and fully described in, for example, U.S. Patent Application Publication 20170145065 and Merrifield, 1963, J. Am. Chem. Soc., vol. 85, pages 2149-2154, which are incorporated by reference herein. Peptides may be isolated from the reaction mixture by chromatographic methods, such as reverse-phase HPLC.

In another embodiment, the peptides of the invention the peptides of the invention are produced recombinantly by methods well known in the art. For example, peptides may be produced by recombinant DNA techniques by cloning and expressing within a host micro-organism or cell a DNA fragment carrying a nucleic acid sequence encoding one of the above-described peptides. Nucleic acid coding sequences can be prepared synthetically, or may be derived from existing nucleic acid sequences (e.g. the sequence coding for wild-type CATH2) by site-directed mutagenesis. These nucleic acid sequences may then be cloned in a suitable expression vector and transformed or transfected into a suitable host cell, such as Escherichia coli, Bacillus spp, Lactobacillus spp, Streptomyces spp, mammalian cells (such as CHO, HEK or COS-1 cells), yeasts (e.g. Saccharomyces, Schizophyllum), insect cells or viral expression systems, such as baculovirus systems, or plant cells. Techniques of constructing and expressing the nucleic acids are well known to a person skilled in the art.

Peptides can be isolated from the culture of the host cells. This can be achieved by common protein purification and isolation techniques, which are available in the art. Such techniques may e.g. involve immunoadsorption or chromatography. Peptides can also be provided with a tag (such as a histidine tag) during synthesis, which allows for a rapid binding and purification, after which the tag is enzymatically removed to obtain the active peptide.

Alternatively, the peptides can be produced in cell-free systems, such as the Expressway cell-free system of Invitrogen.

### Pharmaceutical Compositions

In another embodiment, provided herein is a pharmaceutical composition to treat an inactivated innate immune memory or its associated disease in a subject, the composition comprising: a therapeutically effective amount of CATH2 peptide or a variant thereof, wherein said CATH2 peptide or said variant thereof is present in an amount effective to treat said inactivated innate immune memory or its associated disease.

The invention also provides a pharmaceutical composition comprising the peptide of the invention and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions of the invention may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable or infusible use, for example, intra-mammary injectable or infusible use, include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections or infusions are processed, filled into containers such as ampoules, bags, bottles, syringes or vials, and sealed under aseptic conditions according to methods known in the art. Further, the preparations may be packaged and sold in the form of a kit such as those described in US Appl. Publ. No. 2002/0102208 A1, which is incorporated herein by reference in its entirety. Such articles of manufacture will preferably have labels or package inserts indicating that the associated compositions are useful for treating a subject suffering from, or predisposed to an inactivated innate immune memory associated diseases or disorders.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a non-human mammal (e.g., a cow), but humans can also be treated. Treatment dosages may be titrated using routine methods known to those of skill in the art to optimize safety and efficacy.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual (e.g., animal), and the ability of the molecule to elicit a desired response in the individual. A therapeutically effective amount is also one in which any toxic or detrimental effects of the molecule are outweighed by the therapeutically beneficial effects.

The invention further provides a kit comprising a therapeutically effective amount of a CATH2 peptide, or a derivative thereof.

The invention further provides methods of treating a disease or condition, comprising administering to a mammal in need thereof a therapeutically effective amount of a CATH2 peptide, or a derivative thereof.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

In one aspect, the invention provides a method for treating a condition of an inactivated innate immune memory in a bovine subject, the method comprising: administering to said bovine an effective amount of cathelicidin 2 (CATH2) peptide or a variant thereof, thereby treating said condition of the inactivated innate immune memory in said subject.

Innate immune memory of the invention may depend on a group of proteins, phagocytic cells, non-phagocytic cells, or a combination thereof. Examples of phagocytic cells include, for example, monocytes, macrophages, neutrophils, dendritic cells, mast cells, or a combination thereof. Examples of non-phagocytic cells include, for example, NK cells, epithelial cells, or a combination thereof.

Bovine innate immune memory of the invention may be associated with any of the pathogenic diseases in bovine. Activating bovine innate immune memory of the invention may treat these diseases. Examples of these diseases include, for example, but not limited to, mastitis, metritis, bovine respiratory disease, or other bovine inflammatory diseases.

In a particular embodiment, bovine innate immune memory of the invention is associated with mastitis.

In some embodiments, bovine innate immune memory of the invention is associated with one or more pathogens, including, for example, but not limited to, E. coli, Klebsiella spp., Enterobacter spp., Salmonella spp., Citrobacter spp., Serratia spp., Shigella spp., Edwardsiella spp., Hafnia spp., Morganella spp., Providencia spp., Yersinia spp., Staphylococcus aureus, Staphylococcus spp., Pseudomonas spp., Streptococcus agalactiae, Streptococcus dysgalactiae, Streptococcus uberis, Streptococcus spp., Enterococci, Corynebacterium spp., Arcanobacterium spp., Actinomyces spp., Mycobacterium spp., Prototheca spp., Mycoplasma spp., and Erwinia spp.

More than one agent may be administered, either incorporated into the same composition or administered as separate compositions.

The peptide of the invention may be administered alone, or in combination with one or more therapeutically effective agents (e.g., an antibiotic, another immunomodulator, another cathelicidin, or a combination thereof) or treatments. The other therapeutically effective agent may be conjugated to the peptide of the invention, incorporated into the same composition as the peptide of the invention, or may be administered as a separate composition. The other therapeutically agent or treatment may be administered prior to, during and/or after the administration of the peptide of the invention.

In one embodiment, the peptide of the invention is co-administered with another therapeutic agent. In another embodiment, the peptide of the invention is administered independently from the administration of another therapeutic agent. In one embodiment, the peptide of the invention is administered first, followed by the administration of another therapeutic agent. In another embodiment, another therapeutic agent is administered first, followed by the administration of the peptide of the invention.

The administration of the peptide of the invention with other agents and/or treatments may occur simultaneously, or separately, via the same or different route, at the same or different times. Dosage regimens may be adjusted to provide the optimum desired response (e.g., a therapeutic or prophylactic response).

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, parenteral (including intramammary, subcutaneous, intravenous, intramedullary, intraarticular, intramuscular, or intraperitoneal injection) rectal, topical, transdermal or oral (for example, in capsules, suspensions or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

The composition of the invention (e.g., CATH2 peptide) may be administered parenterally (e.g., intramammary, intravenous, subcutaneous, intraperitoneal, intramuscular). In a particular embodiment, the composition of the invention is administered by intramammary infusion or injection.

In another aspect, the invention provides an intra-mammary delivery composition comprising: CATH2 peptide or a variant thereof. In one example, the peptide or its variant is present in the composition in an amount effective to activate or induce an innate immune memory of the invention in a subject.

The composition of the invention may also be administered by intramuscular or subcutaneous injection. In some embodiments, the composition of the invention may be administered orally. As used herein, a “composition” refers to any composition that contains a pharmaceutically effective amount of one or more active ingredients (e.g., a CATH2 peptide or a derivative thereof).

In yet another aspect, the invention provides a kit or a mammary delivery device comprising: a chamber for storing a composition, wherein said composition comprises CATH2 peptide or a variant thereof.

Mammary delivery devices, including intra-mammary delivery devices, are well known in the art. In one embodiment, the device of the invention is an intra-mammary infusion device. In another embodiment, the device of the invention is a syringe. In yet another embodiment, the device of the invention is a teat-sealant device.

The inventions described herein can be used to treat any suitable mammal, including primates, such as bovine (e.g., cow, buffalo, bison, yak), swine, goat, sheep, horses, cats, dogs, monkeys, humans, rabbits, and rodents such as rats and mice. In one embodiment, the mammal to be treated is bovine. In one example, the bovine of the invention is a dairy cow including, for example, lactating cow and dry cow. In another example, the bovine of the invention is a beef cattle.

In one embodiment, the bovine of the invention lacks the ability to activate or induce innate immune memory. In another embodiment, the bovine of the invention has an inactivated innate immune memory. In some embodiments, the bovine of the invention is need of an activated or induced innate immune memory in order to provide an immunity against a pathogen, a disease or a condition.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

### CATH2 Effectively Induces Innate Immune Training in Bovine

The inventors of the instant application provide evidence that CATH2 is capable of inducing a unique innate immune training profile in bovine macrophages. The inventors demonstrate CATH2 treatment in fully differentiated bovine monocyte-derived macrophages followed by removal of CATH2 and a 3-day resting period changes the epigenetic landscape as observed by increased H3 histone modifications and differential microRNA profiles. These epigenetic changes correlate with changes in gene expression in resting cells and macrophage activation to secondary stimulation with TLR4 agonist lipopolysaccharide (LPS) as observed with enhanced lactate production/glycolytic activity, surface marker expression (MHC II, CD14, CD163, CD80), inflammatory/immune gene expression, and cytokine production (IL-1β, IL-6, IL-8/CXCL8, IL-10, IL-12p40, TNFα). In addition to in vitro evidence of innate immune training, the inventors demonstrate similar effects in vivo with intramammary treatments of CATH2 administered prior to intramammary infectious challenge showing enhanced macrophage activation compared to vehicle controls as evidence of enhanced expression of surface marker expression (MHC II, CD14, CD163, CD80) on the milk macrophage population.

### Methods

### Bovine Monocyte-Derived Macrophage Culture

Peripheral blood mononuclear cells (PBMC) were isolated from bovine blood using ficoll gradients. Bovine monocytes were isolated from PBMC using magnetic assisted cell sorting to positively select for CD14+ cells using anti-human CD14 microbeads (Miltenyi Biotec). Purified bovine blood CD14+ monocytes were differentiated into macrophages using an M2-skewing differentiation factor (recombinant bovine M-CSF) over 7 days then exposed to CATH2 or a vehicle control in the presence of bovine casein to mimic aspects of the mammary gland for 24 hr. Following treatment, vehicle and drug were removed and cells were washed to eliminate residual drug and monocyte-derived macrophages were rested for 3 days in order to assess reprogramming. Following the rest period, a subset of vehicle- and CATH2-trained monocyte-derived macrophages were assessed for epigenetic and transcriptomic changes. The remaining cells were challenged with LPS or E. coli bioparticle labeled with pH-sensitive pHrodo green dye and key parameters for macrophage activation/immune response were assessed including glycolytic activity, surface marker expression, gene expression, cytokine production, phagocytosis, reactive nitrogen species production, and miRNA profiling.

In Vivo Intramammary Treatment & E. coli Mastitis Challenge Model

Healthy lactating dairy cows were treated according to table 1 on day −2 of study, at the AM milking, in the front quarters designated by the allotment, one quarter was treated with CATH2 and the neighboring quarter within the same cow was given vehicle control by intramammary infusion. Following administration of CATH2 and vehicle doses, quarters which received CATH2 or vehicle received an intramammary infusion of live Escherichia coli. The E. coli strain used causes clinical mastitis. Milk samples from CATH2-treated and vehicle control quarters were collected following E. coli challenge for cell isolation to assess macrophage surface marker expression.

TABLE 1
In vivo treatment design for CATH in healthy dairy cows prior to E. coli challenge.

| | Challenge | Treatment | | Route of |
|---|---|---|---|---|
| Treatment | Administration | Description/Dose | Dosage | Administration |
| T01 | Escherichia coli, | 100 mg of | 4 total doses, AM and | Intramammary |
| | Day 0 AM | CATH2/10 mL | PM Day −2 and Day −1 | infusion |
| T02 | (Intramammary | 400 mg of | 1 total dose, AM Day −2 | |
| | infusion) | CATH2/10 mL | | |
| T03 | | Vehicle control | 4 total doses, AM and | |
| | | | PM Day −2 and Day −1 | |
| T04 | | Vehicle control | 1 total dose, AM Day −2 | |

T01 and T03 were administered neighboring right and left quarters within a cow and T02 and T04 were administered neighboring right and left quarters within a cow i.e. treatment and vehicle control were paired within cow.

Phagocytosis & Uptake of pHrodo Green-Labeled E. coli Bioparticle

In brief, E. coli bioparticle labeled with pHrodo-green pH sensitive dye (Invitrogen) were added to monocyte-derived macrophages and phase and green fluorescence was captured over time using the S3 IncuCyte® Live-Cell Analysis System (Essen Bioscience) over a period of 24-hr. Five images were captured per well, and images were analyzed using IncuCyte® analysis software to apply appropriate masking to quantify both area per image and intensity of green fluorescence. As bioparticle is taken up by macrophages and phagolysosome matures the pH which the bioparticle is exposed to decreases which causes the pH-sensitive dye to fluoresce, the intensity of the fluorescent signal increases with decreasing pH.

Reactive Nitrogen Species Measurements from Bovine Monocyte-Derived Macrophage Culture Supernatant

Nitrite, a primary breakdown product of nitric oxide, in cell culture supernatant were quantified using Griess Reagent System (Promega) according to manufacturer's instructions.

### Cytokine Analysis of Monocyte-Derived Macrophages

Cytokine levels in cell culture supernatant (IL-8/CXCL8, IL-6, TNFα, IL-1β, IL-10, IL-12p40) were quantified using customized U-Plex assays (MesoScale Diagnostics, LLC). Multiplex (IL-8/CXCL8, IL-6, TNFα, IL-1β, IL-10) and single-plex (IL-12p40) assays were developed according to manufacturer's recommendations. In brief, biotinylated antibodies specific to cytokines of interest were linked to specific MSD Linkers (Meso Scale Diagnostics, LLC) according to manufacturer's recommendations and used as capture antibodies for coating U-Plex plates. After U-Plex plates were coated with capture antibodies, culture supernatant samples and reference recombinant proteins were added to plates. Captured cytokines were detected with sulfotagged detection antibody solutions (antibodies used for detection were conjugated with MSD SULFO-TAG (Meso Scale Diagnostics, LLC, Rockville, MD) according to manufacturer). Following incubation with detection antibodies, plates were washed, and MSD Read Buffer (Meso Scale Diagnostics, LLC, Rockville, MD) was added and data were captured on a MESO SECTOR S 600 MM instrument (Meso Scale Diagnostics, LLC, Rockville, MD). Raw data were analyzed using MSD DISCOVERY BENCHWORK.

Lactate Measurements from Bovine Monocyte-Derived Macrophage Culture Supernatant

Lactate levels in cell culture supernatant were quantified using LactateGlo® (Promega) according to manufacturer's instructions.

### Histone Modifications (Quantification in Bovine Monocyte-Derived Macrophages)

Histones were isolated from bovine monocyte-derived macrophages using commercially available histone extraction kits (Epigentek) according to the manufacturer's instructions and stored at −80° C. until histone quantification assays could be performed. Total H3 histones and three histone modifications (H3K27ac, H3K4me1, H3K36me3) were quantified using commercially available ELISA kits (Epigentek) according to the manufacturer's instructions. Data was captured using a SpectraMax (Molecular Devices).

### Histone Modifications—Chromatin Immunoprecipitation Sequencing (ChIP-Seq) for H3K27ac in Bovine Monocyte-Derived Macrophages

Chromatin was isolated from bovine monocyte-derived macrophages and subsequent chromatin immunoprecipitation sequencing (ChIP-Seq) to identify genome-wide DNA-H3K27ac histone modification interactions. Peak calling analysis was performed sing Epic2 with optimized parameters for H3K27ac. ChIP-sequencing and peak calling analysis to assess H327ac was performed by Diagenode (Denville, NJ 07834 United States & 4102 Seraing (Ougrde) Belgium).

Flow Cytometric Analysis of Surface Marker Expression on Bovine Monocyte-Derived Macrophages and Cells Isolated from Bovine Milk

Expression of surface markers on bovine monocyte-derived macrophages and cells isolated from bovine milk were assessed using flow cytometry. In brief, for monocyte-derived macrophages, cells were lifted with Accutase, washed, and then incubated with fluorescently tagged antibodies against bovine surface markers of interest (CD163, CD14, CD80, MHC II) and a live/dead stain. In brief, for cells in bovine milk, milk was diluted in MACS Running Buffer, spun, fat layer and supernatant were discarded, and milk cell pellets were preserved in Streck. Preserved milk cells were washed again and then incubated with fluorescently tagged antibodies against bovine surface markers of interest (CD163, CD14, CD80, MHC II, CD172a) and a live/dead stain. Following incubation with live/dead stain and antibodies, labeled cells were washed, and then analyzed on a ZE5 CellAnalyzer (BioRad) for monocyte-derived macrophages and a FACS Canto II (BD) for milk cells. Flow cytometry data were analyzed using FlowJo analysis software (Becton Dickson) to assess relative levels of expression via mean fluorescent intensity for each marker of interest within the live CD14+ cell population (for monocyte-derived macrophages) or within the live CD14+CD172a+ cell population (for milk cells).

Transcriptomic Analysis (microRNASeq and mRNASeq) of Bovine Monocyte-Derived Macrophages

Bovine monocyte-derived macrophages were lysed in Buffer RLT with addition of (3-mercaptoethanol (QIAGEN) and stored at −80° C. until miRNA/mRNA isolations could be performed. Total RNA including miRNA was isolated from cell lysates using the miRNeasy Tissues/Cells Advanced kit (QIAGEN) following the manufacturer's instructions. mRNA and miRNA libraries were prepared using the TruSeq Stranded mRNA Library Prep Kit (Illumina) and QIAseq miRNA Library Kit, respectively (QIAGEN) according to the manufacturer's instructions. Libraries were run on the Illumina NextSeq550 System. Sequencing data were analyzed using CLC Genomics Workbench software and QIAGEN Ingenuity Pathway Analysis (IPA).

### Results

### Cath2 Enhances LPS-Induced Cytokine Response in Bovine Monocyte-Derived Macrophages

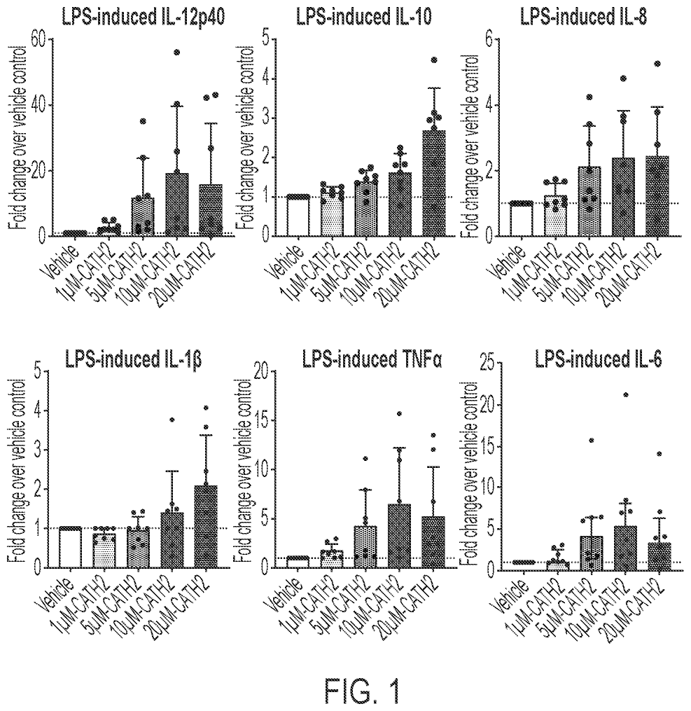

Exposure of bovine monocyte-derived macrophages to CATH2 resulted in enhanced the cytokine responses to LPS challenge and enhanced production of both pro- and anti-inflammatory cytokines/chemokines including IL-12p40, IL-6, TNFα, IL-8, IL-1β, and IL-10 (FIG. 1). Increased pro-inflammatory cytokine responses to secondary stimuli such as LPS have been observed with other stimuli known to drive innate immune training. Enhanced production of anti-inflammatory cytokine IL-10 with CATH2-training indicates CATH2 is capable of generating a unique macrophage reprogramming profile with both enhanced inflammatory responses (typical of M1-like macrophages) as well as regulatory responses (typical of M2-like macrophages).

### CATH2 Enhances LPS-Induced Lactate Production in Bovine Monocyte-Derived Macrophages

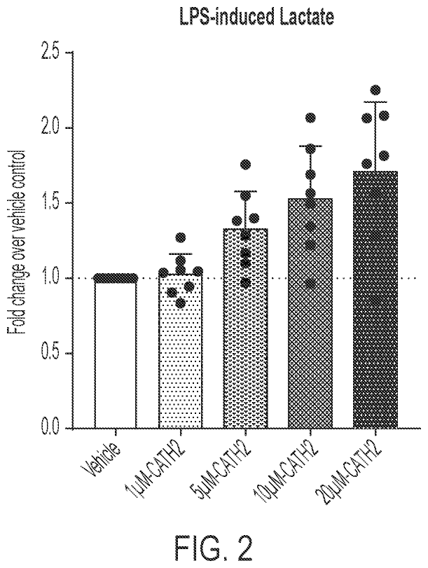

Immunometabolic pathways are important for both innate training and responses to inflammatory stimuli like LPS. Changes in oxygen and glucose consumption necessitate the metabolic requirements of activated macrophages to carry out immunological functions. In addition to the direct role in immune response, metabolic intermediates also influence epigenetic reprogramming by influencing activity of epigenetic enzymes like histone demethylases or by acting as acetyl donors. Priming through exposure to CATH2 prior to LPS challenge resulted in altered glycolytic activity in bovine monocyte-derived macrophages as observed by increased lactate production measured in cell culture supernatant (FIG. 2). Increased glycolytic activity has been observed with other stimuli known to drive innate immune training.

### CATH2 Alters Histone Modification Profiles in Bovine Monocyte-Derived Macrophages

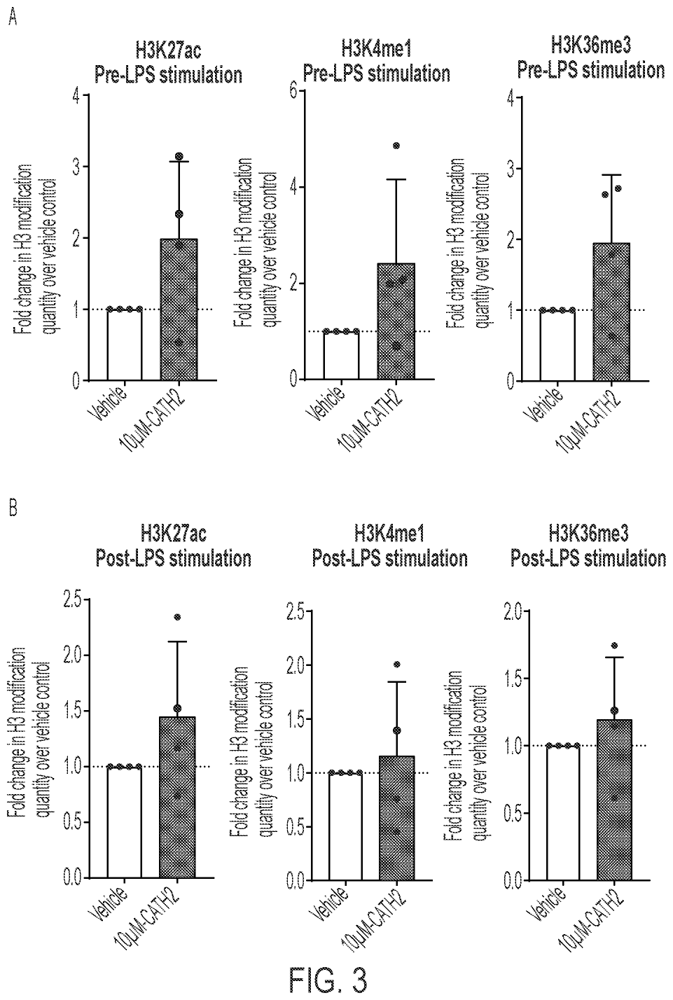

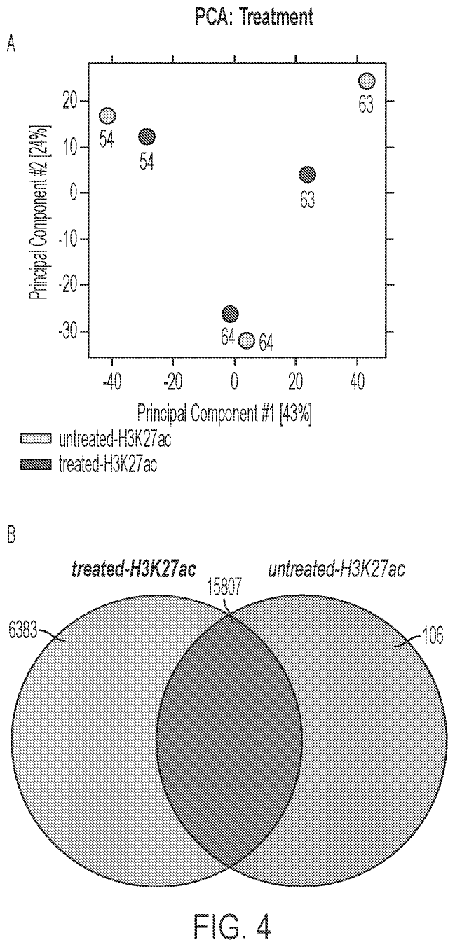

During initiation of innate immune training, stimulation of innate immune cells leads to modifications in histone proteins and DNA methylation status, leading to unfolding of chromatin and facilitating transcription and expression of immune factors such as pro-inflammatory cytokines. Ultimately, these types of epigenetics changes drive faster enhanced recruitment of transcription factors and subsequent gene expression following challenge with secondary stimuli like LPS. The acquisition of histone 3 lysine 27 acetylation (H3K27ac) marks at distal enhancers marked with histone 3 lysine 4 methylation (H3K4me1) and the consolidation of histone 3 lysine 4 trimethylation (H3K4me3) marks at the promoters of stimulated genes have been shown to be key hallmarks of epigenetic reprogramming associated with innate immune training. Here the inventors show bovine monocyte-derived macrophages exposed to CATH2 acquire increased levels of various H3 histone modifications following a rest period and some modifications are retained to a lesser extent following LPS challenge, this includes key innate immune training-relevant histone marks H3K27ac and H3K4me1 (FIG. 3). Chromatin immunoprecipitation sequencing (ChIP-Seq) of H3K27ac in monocyte-derived macrophages exposed to CATH2 also show increased number of peaks with H3K27ac mark compared to monocyte-derived macrophages exposed to vehicle control (FIG. 4). These data indicate immunological reprogramming driven by CATH2 in bovine cells have underpinnings of epigenetic reprogramming and specifically have increases in marks associated with innate immune training (FIG. 3, FIG. 4).

### CATH2 Alters Surface Marker Expression in Bovine Monocyte-Derived Macrophages and Milk Macrophages Following Challenge (Flow Cytometric Analysis)

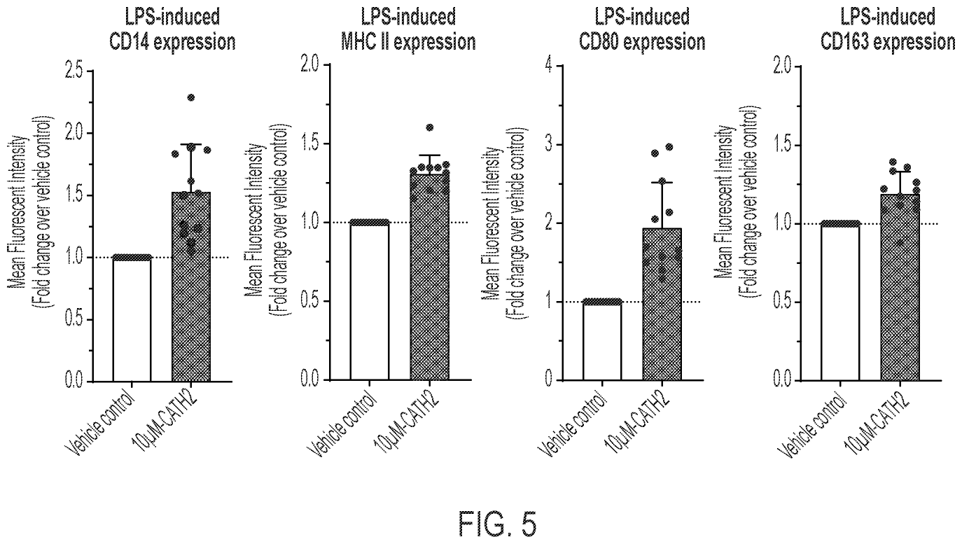

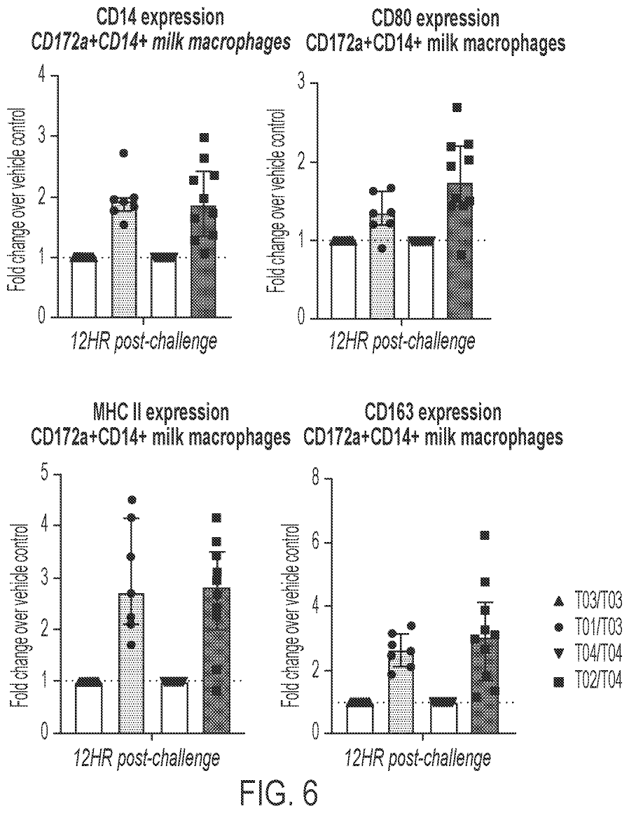

In vitro CATH2 treatment enhanced expression of multiple surface markers associated with macrophage activation including CD14 (co-receptor for TLR4 signaling), CD80 (co-stimulatory molecular for T cell activation), MHC II (required for antigen presentation to T cells), and CD163 (high affinity scavenger receptor for the hemoglobin-haptoglobin complex, also known to play a role in sensing gram-negative and gram-positive bacteria) 24 hr following LPS challenge after removal of CATH2 and following a 3-day rest period in bovine monocyte-derived macrophages (FIG. 5). In vivo intramammary treatment of CATH2 prior to bacterial challenge also demonstrated enhanced expression of the same surface markers associated with macrophage activation (CD14, CD80, MHC II, and CD163) 12 hr following live E. coli intramammary challenge in milk macrophages (FIG. 6). Here the inventors provide correlative in vitro and in vivo evidence of innate immune training in macrophage populations by CATH2 treatment prior to stimulation/challenge. Enhanced macrophage activation observed in milk cell populations correlated with better clinical outcomes including reduced bacterial counts in milk, lower milk somatic cell counts, etc.

CATH2 Modifies miRNA Profile in Monocyte-Derived Macrophages (microRNASeq Analysis)

CATH2 was shown to alter miRNA profiles in bovine monocyte-derived macrophages providing evidence of epigenetic reprogramming associated with innate immune training. MicroRNA profiles were assessed following CATH2 or vehicle control exposure and rest period prior to LPS challenge to evaluate effects on resting macrophages. Prior to LPS challenge, 51 miRNAs were significantly altered due to CATH2 training (Table 2). A number of these miRNAs are reported as responsive to inflammatory stimuli and/or play a role in regulating the inflammatory response and polarization of macrophage. Notably, miR-155 was overexpressed and has a well-described role in pro-inflammatory responses and polarization of macrophages towards an M1 phenotype. However, several of the differentially expressed miRNAs have been shown to have opposing roles in macrophage polarization. Therefore, CATH2 macrophage reprogramming in bovine cells is likely more complex than the two simplified M1 and M2 subtypes.

TABLE 2
Differentially expressed miRNAs following CATH2 treatment
and 3-day rest period prior to LPS challenge.

| | Max | | Log | | |
|---|---|---|---|---|---|
| | group | Fold | fold | | FDR |
| Name | means | change | change | P-value | p-value |
| bta-miR-148a | 38582.5 | −1.78 | −0.83 | 0.0000 | 0.0000 |
| hsa-miR-10395-3p | 55.8 | −2.92 | −1.55 | 0.0000 | 0.0000 |
| bta-miR-184 | 125.3 | 2.61 | 1.38 | 0.0000 | 0.0001 |
| bta-miR-320a | 1483.8 | 1.68 | 0.75 | 0.0000 | 0.0001 |
| bta-miR-365-3p | 1299.3 | 1.59 | 0.67 | 0.0000 | 0.0001 |
| bta-miR-374a | 1353.0 | −1.59 | −0.67 | 0.0000 | 0.0003 |
| hsa-miR-12135 | 207.8 | −1.63 | −0.71 | 0.0000 | 0.0003 |
| bta-miR-1260b | 264.8 | 1.63 | 0.70 | 0.0000 | 0.0004 |
| bta-miR-193a-5p | 976.8 | 1.60 | 0.68 | 0.0000 | 0.0004 |
| hsa-let-7d-3p | 198.8 | 1.62 | 0.70 | 0.0000 | 0.0004 |
| hsa-miR-142-3p | 67909.8 | −1.57 | −0.65 | 0.0000 | 0.0004 |
| hsa-miR-142-5p | 231.0 | −1.55 | −0.63 | 0.0000 | 0.0013 |
| hsa-miR-340-5p | 2980.3 | −1.47 | −0.56 | 0.0000 | 0.0013 |
| hsa-miR-532-3p | 486.0 | 1.51 | 0.60 | 0.0000 | 0.0013 |
| bta-miR-1307 | 1380.8 | 1.49 | 0.57 | 0.0001 | 0.0017 |
| bta-miR-148b | 2107.8 | −1.43 | −0.51 | 0.0001 | 0.0020 |
| hsa-miR-16-1-3p | 17.5 | −2.57 | −1.36 | 0.0001 | 0.0020 |
| bta-miR-101 | 1862.5 | −1.47 | −0.55 | 0.0001 | 0.0028 |
| bta-miR-326 | 841.8 | 1.47 | 0.55 | 0.0002 | 0.0038 |
| hsa-miR-1260b | 42.3 | 2.19 | 1.13 | 0.0002 | 0.0038 |
| bta-miR-2285bf | 791.5 | −1.52 | −0.61 | 0.0002 | 0.0038 |
| bta-miR-29c | 1775.3 | −1.45 | −0.54 | 0.0002 | 0.0038 |
| bta-miR-142-5p | 2320.0 | −1.43 | −0.51 | 0.0002 | 0.0042 |
| bta-miR-1249 | 224.0 | 1.43 | 0.52 | 0.0004 | 0.0073 |
| bta-miR-2447 | 35.3 | 1.92 | 0.94 | 0.0005 | 0.0079 |
| bta-miR-652 | 455.5 | 1.46 | 0.54 | 0.0005 | 0.0079 |
| hsa-miR-30e-5p | 793.5 | −1.45 | −0.54 | 0.0005 | 0.0080 |
| bta-miR-500 | 324.0 | 1.43 | 0.51 | 0.0007 | 0.0103 |
| bta-miR-877 | 44.0 | 1.93 | 0.95 | 0.0007 | 0.0103 |
| bta-miR-149-5p | 586.3 | 1.43 | 0.52 | 0.0009 | 0.0113 |
| bta-miR-30f | 99.5 | 1.57 | 0.65 | 0.0009 | 0.0113 |
| hsa-miR-192-5p | 170.3 | −1.47 | −0.56 | 0.0009 | 0.0113 |
| bta-miR-331-3p | 130.3 | 1.61 | 0.69 | 0.0011 | 0.0130 |
| hsa-miR-21-5p | 197397.5 | −1.33 | −0.41 | 0.0011 | 0.0134 |
| hsa-miR-29b-1-5p | 43.5 | 1.90 | 0.92 | 0.0012 | 0.0140 |
| bta-miR-199a-3p | 113.0 | 1.92 | 0.94 | 0.0014 | 0.0161 |
| bta-miR-187 | 35.8 | −2.07 | −1.05 | 0.0016 | 0.0171 |
| bta-miR-6529a | 4376.5 | 1.32 | 0.40 | 0.0016 | 0.0171 |
| hsa-miR-9983-3p | 448.3 | −1.40 | 0.48 | 0.0016 | 0.0171 |
| bta-miR-181b | 1201.5 | 1.38 | 0.47 | 0.0019 | 0.0197 |
| bta-miR-29a | 5801.0 | −1.36 | −0.44 | 0.0021 | 0.0211 |
| hsa-miR-574-3p | 941.3 | 1.36 | 0.44 | 0.0023 | 0.0218 |
| bta-miR-6523a | 190.8 | 1.39 | 0.47 | 0.0024 | 0.0225 |
| hsa-miR-155-5p | 2323.5 | 1.31 | 0.39 | 0.0034 | 0.0313 |
| hsa-miR-29a-3p | 5740.8 | −1.32 | 0.40 | 0.0041 | 0.0368 |
| hsa-miR-152-3p | 336.8 | −1.37 | 0.45 | 0.0043 | 0.0375 |
| bta-miR-27a-3p | 1731.5 | −1.31 | −0.39 | 0.0047 | 0.0402 |
| hsa-miR-143-3p | 30.8 | −1.96 | −0.97 | 0.0048 | 0.0404 |
| bta-miR-197 | 272.0 | 1.37 | 0.46 | 0.0049 | 0.0409 |
| bta-miR-92b | 148.0 | 1.52 | 0.60 | 0.0054 | 0.0437 |
| bta-miR-296-5p | 14.3 | 2.10 | 1.07 | 0.0058 | 0.0464 |

CATH2-training also resulted in differential expression of miRNA following LPS challenge (Table tables 3 & 4). At 8 hours post LPS challenge, miR-155 remained higher than vehicle control as we observed prior to LPS challenge (Table 2). miR-146b was significantly different at both the 8- and 24-hour following LPS challenge. miR-155 was consistently upregulated prior to and following LPS stimulation, indicating CATH2 induces innate immune training to alter the responses to inflammatory insults like LPS both prior to and following challenge. MicroR-155 and miR-146a/b expression in human cells is coordinately regulated via NFKB signaling and are simultaneously induced by LPS treatment. While miR-155 is known to potentiate the inflammatory response, miR-146a/b participates in a negative feedback loop for NFKB signaling. CATH2 treatment of bovine monocyte-derived macrophages also resulted in decreased miR-148a and increased miR-181b levels following resting period bot prior to and 8 hours following LPS challenge. In human and mouse macrophages, miR-148a is more highly expressed in M1-polarized macrophages and actively promotes M1 polarization. In murine RAW264.7 cells, miR-181b is stimulated by LPS and directly targets IL-6 to promote endotoxin tolerance. The function of these bovine miRNA in bovine monocyte/macrophage populations within the context of macrophage polarization and/or innate immune cell reprogramming is not well-defined, but these data provide evidence CATH2 treatment is capable of inducing epigenetic reprogramming in these cells. It is likely that CATH2 reprograms bovine monocyte-derived macrophages to have a unique epigenetic landscape, with miRNA capable of enhancing key inflammatory responses while maintaining regulatory mechanisms leading to enhanced but balanced immune responses.

TABLE 3
Differentially expressed miRNAs following CATH2 treatment
and 3-day rest period 8-hour post-LPS challenge.

| | Experiment A | | | | | Experiment B | | | | |
|---|---|---|---|---|---|---|---|---|---|---|
| | Max | Log₂ | | | FDR | Max | Log₂ | | | FDR |
| | group | fold | Fold | P- | p- | group | fold | Fold | P- | p- |
| Name | mean | change | change | value | value | mean | change | change | value | value |
| hsa-miR-155-5p | 6502.3 | 0.92 | 1.89 | 0.0000 | 0.0000 | 8145.3 | 1.14 | 2.20 | 0.0000 | 0.0000 |
| bta-miR-181b | 1521.5 | 0.68 | 1.60 | 0.0000 | 0.0004 | 1557.5 | 0.82 | 1.76 | 0.0000 | 0.0006 |
| bta-miR-146b | 5381.8 | 1.51 | 2.85 | 0.0000 | 0.0000 | 4366.0 | 0.64 | 1.56 | 0.0008 | 0.0339 |
| bta-miR-148a | 47012.5 | −0.84 | −1.79 | 0.0000 | 0.0000 | 17960.0 | −0.52 | −1.44 | 0.0007 | 0.0332 |
| bta-miR-1814c | 53.3 | −0.83 | −1.77 | 0.0005 | 0.0151 | 29.5 | −1.04 | −2.05 | 0.0001 | 0.0067 |

TABLE 4
Differentially expressed miRNAs following CATH2 treatment
and 3-day rest period 24-hour post-LPS challenge.

| | Experiment A | | | | | Experiment B | | | | |
|---|---|---|---|---|---|---|---|---|---|---|
| | Max | Log₂ | | | FDR | Max | Log₂ | | | FDR |
| | group | fold | Fold | P- | p- | group | fold | Fold | P- | p- |
| Name | mean | change | change | value | value | mean | change | change | value | value |
| bta-miR-146b | 9975.8 | 2.18 | 4.52 | 0.0000 | 0.0000 | 14789.0 | 2.12 | 4.34 | 0.0000 | 0.0000 |
| hsa-miR-146b-5p | 1076.3 | 1.18 | 2.27 | 0.0000 | 0.0012 | 1913.0 | 0.77 | 1.71 | 0.0001 | 0.0019 |

CATH2 Modifies Transcriptome of Bovine Monocyte-Derived Macrophages/Transcriptomic Response Following LPS Challenge in Bovine Monocyte-Derived Macrophages (mRNASeq Analysis)

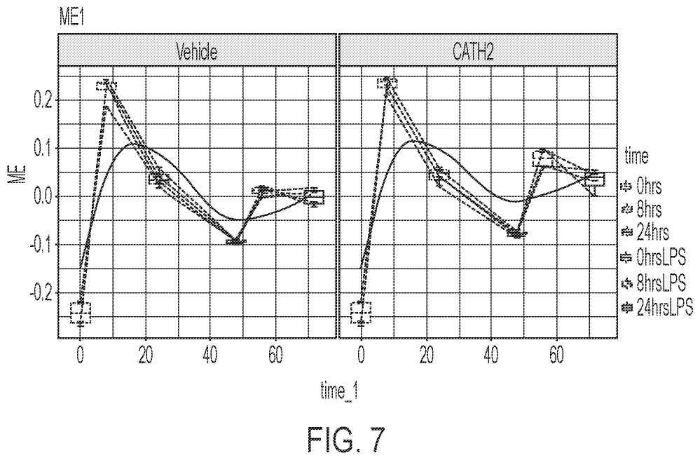

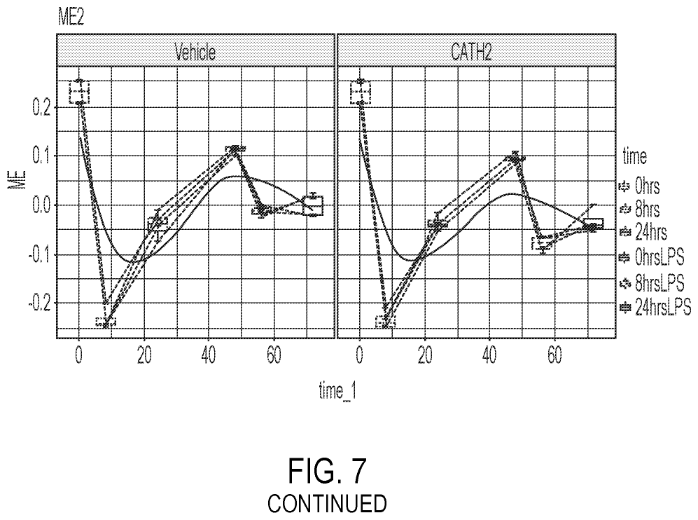

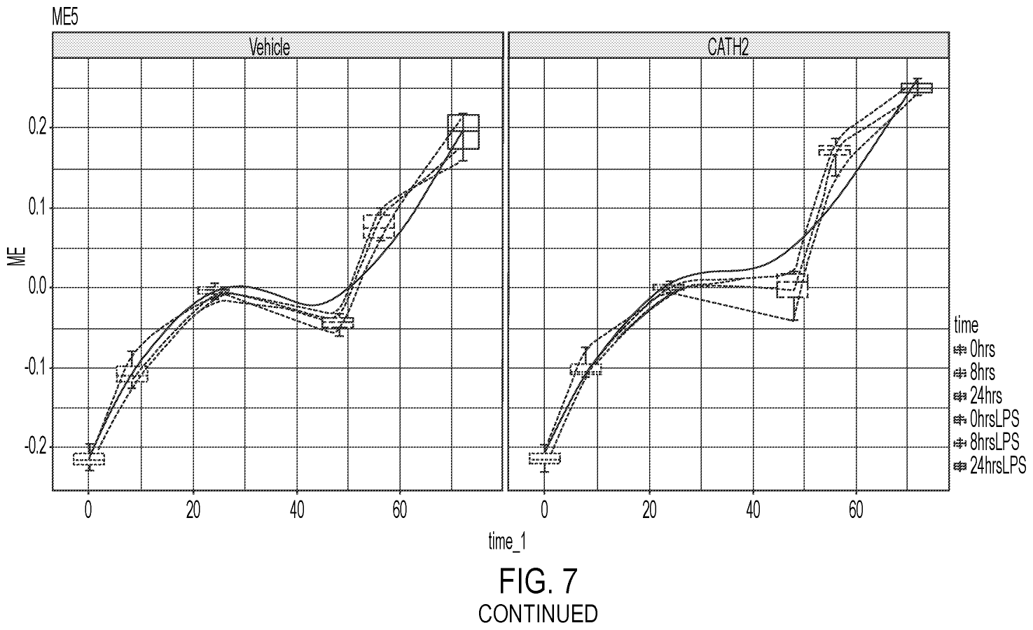

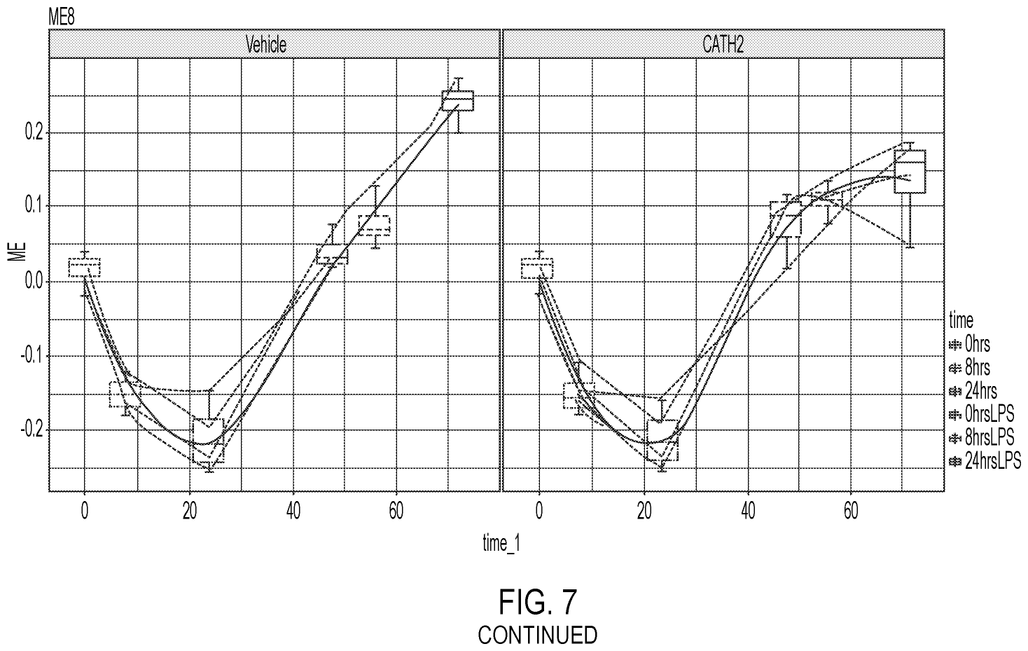

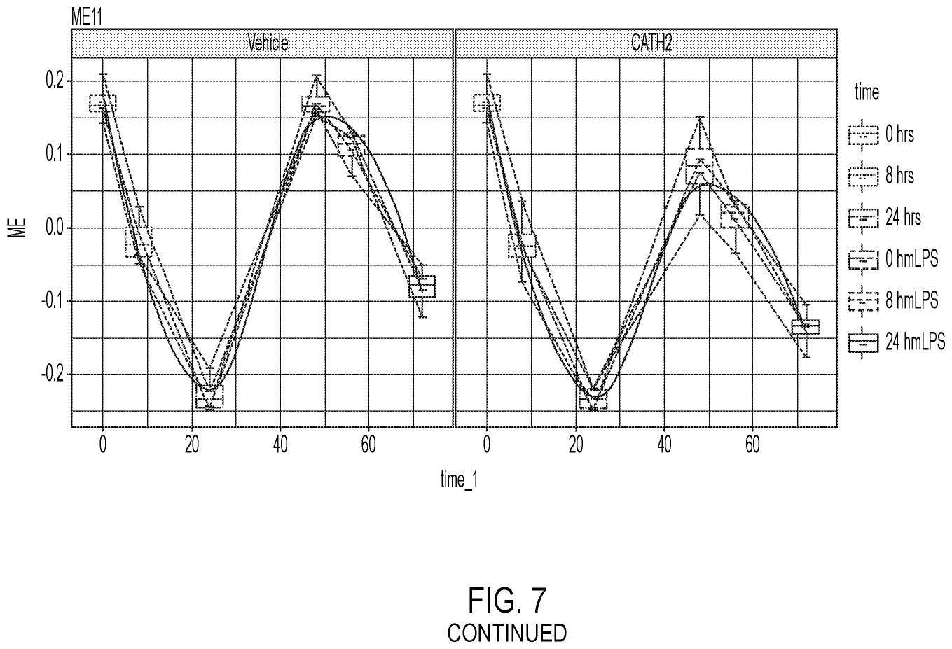

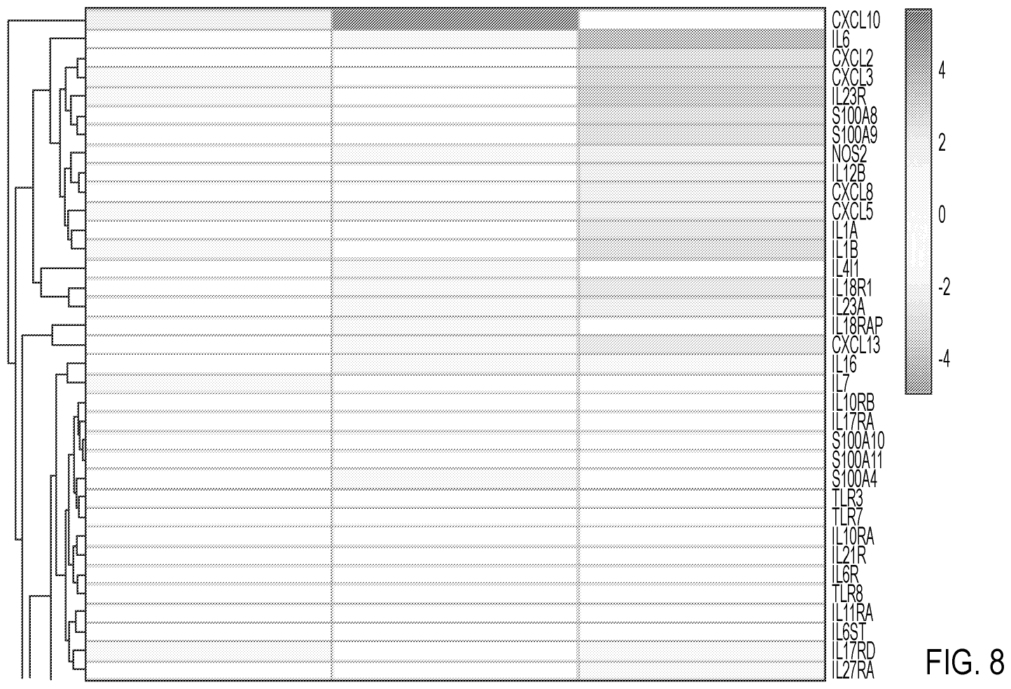

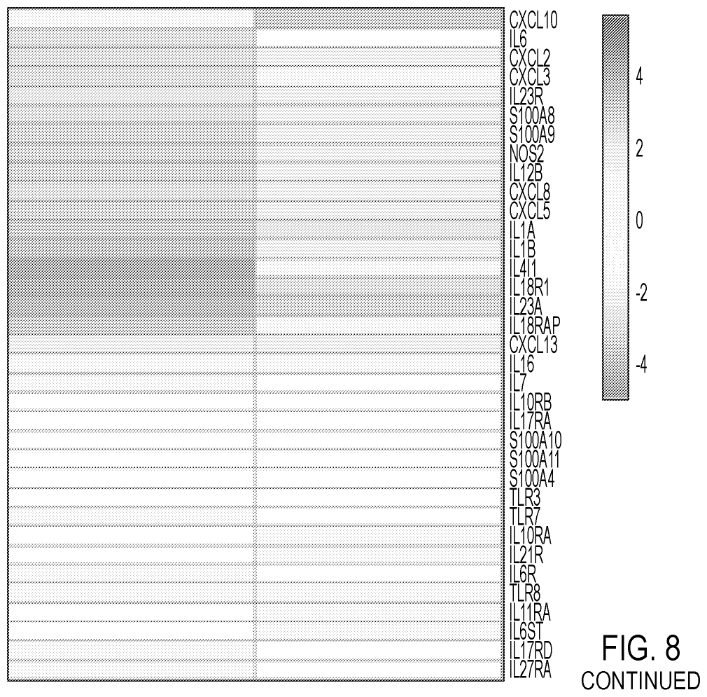

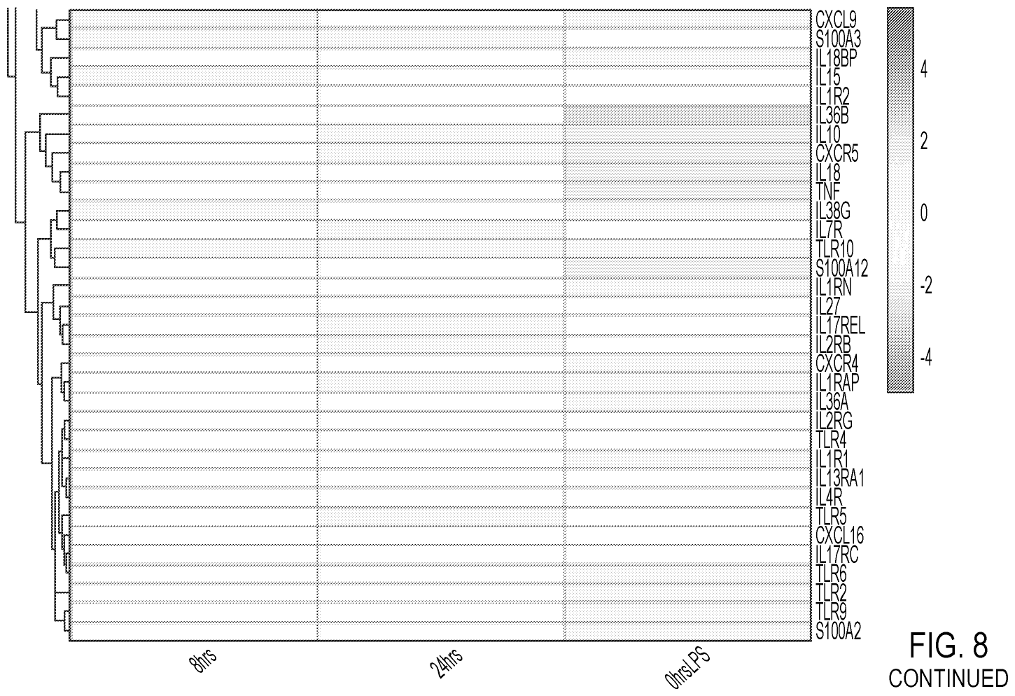

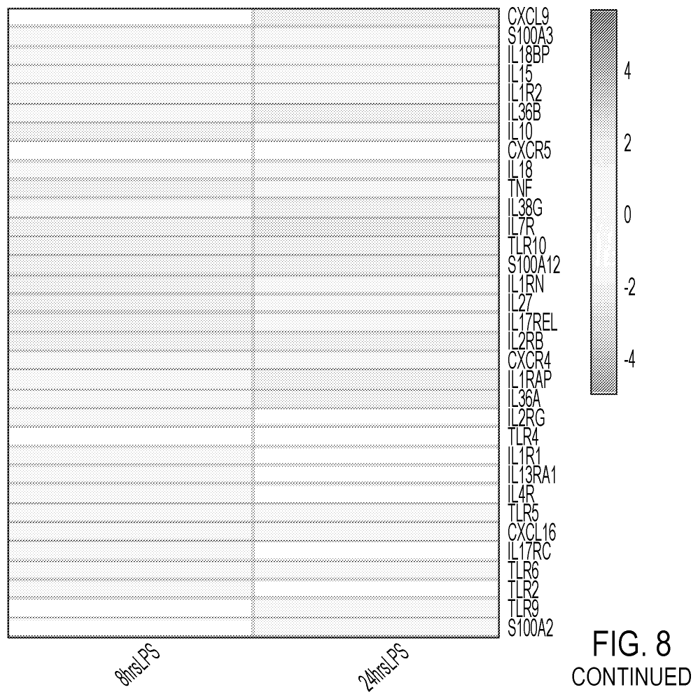

Transcriptome analysis was used to determine CATH2 treatment effect and dynamics on key pathways, including differentially expressed gene (DEG) analysis and the co-expression analysis. The largest relative difference (between CATH2 and vehicle control) was seen at 8 hours following LPS challenge, according to the number of significant DEG (adjusted P<0.05 and absolute log 2(Fold Change)>1; Table 5) and gene module expression pattern (FIG. 7). A considerable number of significant DEG were also identified prior to LPS challenge following CATH2 treatment, removal of CATH2, and a 3-day rest period. The significant DEGs and co-expression modules were used to identify the predicted upstream regulators and pathways associated with treatment.

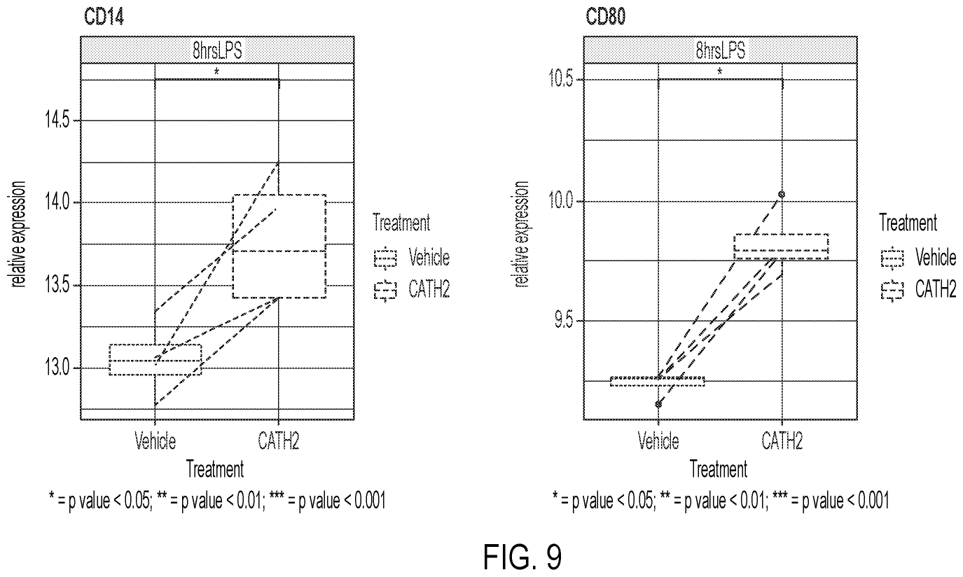

At both pre- and post-LPS stimulation time points following CATH2 treatment and 3-day period of rest, many immune response genes (e.g. cytokines and chemokines) were significantly upregulated in CATH2-treated bovine monocyte-derived macrophages, including IL1A/B, IL6, IL8, IL12, IL23, CCL2, NOS2 and IL10 (FIGS. 7 & 8). Similarly, significant DEGs enriched functions were central to immune response pathways such as IL17, IL6, NFκB, IL23 signaling, TNF, IL1B and interferon signaling (type and type II). They were also significantly associated with functions such as phagocyte and leukocyte stimulation, chemotaxis, and antimicrobial activity. The IL17 signaling pathway was identified as the top pathway associated with CATH2's effect on macrophage supported by significant DEGs, such as IL1B, IL12B, NOS2 and IL23A which drive Th17-type mucosal immunity. In addition, IL-12 and IL-18 can induce IFNγ production that is critical for defense against many bacteria. Many type I interferon induced genes (e.g. ISG15, MX2 and OAS2) were also significantly upregulated in CATH2 treated bovine monocyte-derived macrophages. Many of these pathways and functions were predicted to be more activated in the CATH2 treated bovine monocyte-derived macrophages at both prior to and following LPS challenge. A subset of DEG related Rho family signaling were downregulated in CATH2-treated bovine monocyte-derived macrophages, which could also play roles in macrophage chemotaxis. In addition, macrophage associated surface marker expression (i.e. CD14 and CD80) were significantly upregulated in CATH2 group compare with vehicle group based on our monocyte-derived macrophage RNA sequence data (FIG. 9).

These mRNA sequencing results indicate that CATH2 treatment in bovine monocyte-derived macrophages modulates resting immune cells and their subsequent response to secondary stimuli like LPS via regulation of gene expression. These enhanced responses driven by CATH2 innate immune training suggest CATH2 has the ability to promote host defense against multiple types of infectious pathogens in cattle.

TABLE 5
Number of significantly differentially expressed genes of CATH2 and
control contrast at each time point relative to CATH2 treatment
or LPS stimulation following CATH2 treatment and 3-day rest period.

| | Padj < 0.05 & | Padj < 0.1 & | P < 0.01 & | P < 0.05 & | Padj < 0.05 & |
|---|---|---|---|---|---|
| CATH2_vs_vehicle | \|log2FC\| > 1 | \|log2FC\| > 1 | \|log2FC\| > 1 | \|log2FC\| > 1 | \|log2FC\| > 0.585 |
| 0 hrs_post_CATH2 | 0 | 0 | 0 | 0 | 0 |
| 8 hrs_post_CATH2 | 0 | 0 | 6 | 27 | 0 |
| 24 hrs_post_CATH2 | 0 | 0 | 11 | 28 | 0 |
| 0 hrs_post_LPS | 96 | 134 | 169 | 244 | 171 |
| 8 hrs_post_LPS | 479 | 532 | 509 | 593 | 962 |
| 24 hrs_post_LPS | 279 | 330 | 328 | 393 | 520 |

### CATH2 Enhances Phagocytosis of E. Coli Bioparticle by Bovine Monocyte-Derived Macrophages

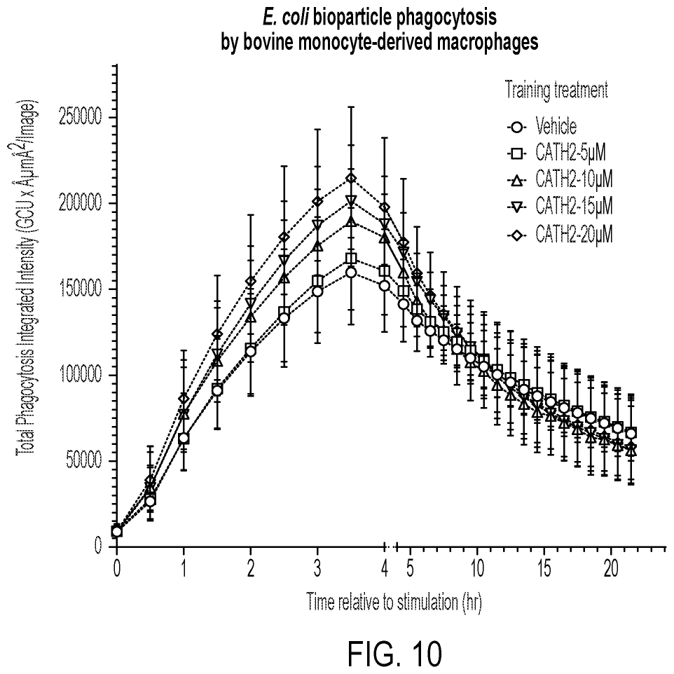

Exposure of bovine monocyte-derived macrophages to CATH2 resulted in enhanced phagocytosis of E. coli bioparticles <4 hr from time of challenge with E. coli bioparticles (FIG. 10). Phagocytosis is required for effective clearance of bacterial pathogens by phagocytes such as macrophages. Early effective bacterial clearance by phagocytes leads to early control of infection and immunopathology of disease.

### CATH2 Enhances E. Coli Bioparticle-Induced RNS in Bovine Monocyte-Derived Macrophages

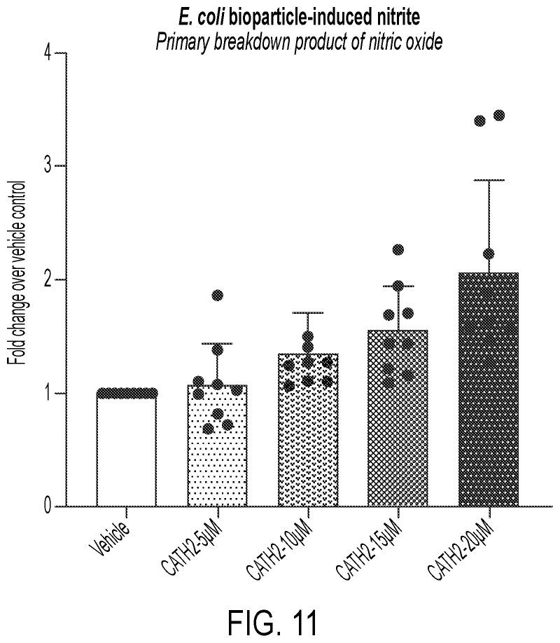

Exposure of bovine monocyte-derived macrophages to CATH2 resulted in enhanced production of nitrogen oxide species/reactive nitrogen species (RNS) responses to E. coli bioparticle challenge (FIG. 11). Nitric oxide species are key bactericidal factors produced by phagocytes to enable the host to kill and clear bacterial infections.

In summary, based on these results, the inventors of the instant application have demonstrated that the CATH2 peptide effectively activates or induces innate immune memory in bovine.

Having described preferred embodiments of the invention, it is to be understood that the invention is not limited to the precise embodiments, and that various changes and modifications may be effected therein by those skilled in the art without departing from the scope or spirit of the invention as defined in the appended claims.

1-54. (canceled)

55. A method for activating or inducing innate immune memory in a bovine in need thereof, the method comprising: administering to said bovine an effective amount of cathelicidin 2 (CATH2) peptide, wherein said peptide comprises the amino acid sequence set forth in SEQ ID NO.: 35, 36, or 37, thereby activating or inducing innate immune memory in said bovine.

56. The method of claim 55, wherein said bovine lacks the ability to activate or induce innate immune memory.

57. The method of claim 55, wherein said bovine has an inactivated innate immune memory.

58. The method of claim 55, wherein said bovine is need of an activated or induced innate immune memory in order to provide an immunity against a pathogen, a disease or a condition.

59. The method of claim 58, wherein said disease is associated with a bacteria, a virus, or another organism.

60. The method of claim 59, wherein said bacteria is E. coli, S. aureus, S. uberis, or a combination thereof.

61. The method of claim 55, wherein said peptide is capable of being effective in a mammary gland.

62. The method of claim 55, wherein said peptide is an immune modulatory peptide.

63. The method of claim 55, wherein the effectiveness of said peptide is induced by immuno-modulation.

64. The method of claim 55, wherein said peptide has no antibiotic activity.

65. The method of claim 60, wherein said peptide has no direct killing effect on said bacteria.

66. The method of claim 55, wherein said administration is an intra-mammary administration.

67. The method of claim 55, wherein said bovine subject is a beef animal.

68. The method of claim 55, wherein said bovine subject is a dairy cow.

69. The method of claim 55, wherein the treatment is an immuno-modulatory treatment.


SEQUENCE LISTING (37 sequences)

SEQ ID NO: 1 - protein, 27 aa, synthetic construct
  REGION 1..27: note = CMAP27
RFGRFLRKIRRFRPKVTITIQGSARFG

SEQ ID NO: 2 - protein, 21 aa, synthetic construct
  REGION 1..21: note = cycCMAP(1-21)[Lys8]
  SITE 8: note = MISC_FEATURE - is Fmoc-L-bishomopropargylglycine
  SITE 21: note = MISC_FEATURE - azido resin attached
RFGRFLRXIRRFRPKVTITIQ

SEQ ID NO: 3 - protein, 21 aa, synthetic construct
  REGION 1..21: note = cycCMAP(1-21)[Arg7]
  SITE 7: note = MISC_FEATURE - is Fmoc-L-bishomopropargylglycine
  SITE 21: note = MISC_FEATURE - azido resin attached
RFGRFLXKIRRFRPKVTITIQ

SEQ ID NO: 4 - protein, 21 aa, synthetic construct
  REGION 1..21: note = cycCMAP(1-21)[Leu6]
  SITE 6: note = MISC_FEATURE - is Fmoc-L-bishomopropargylglycine
  SITE 21: note = MISC_FEATURE - azido resin attached
RFGRFXRKIRRFRPKVTITIQ

SEQ ID NO: 5 - protein, 21 aa, synthetic construct
  REGION 1..21: note = cycCMAP(1-21)[Leu6],Phe2/Trp
  SITE 6: note = MISC_FEATURE - is Fmoc-L-bishomopropargylglycine
  SITE 21: note = MISC_FEATURE - azido resin attached
RWGRFXRKIRRFRPKVTITIQ

SEQ ID NO: 6 - protein, 21 aa, synthetic construct
  REGION 1..21: note = cycCMAP(1-21)[Leu6],Phe2,5/Trp
  SITE 6: note = MISC_FEATURE - is Fmoc-L-bishomopropargylglycine
  SITE 21: note = MISC_FEATURE - azido resin attached
RWGRWXRKIRRFRPKVTITIQ

SEQ ID NO: 7 - protein, 21 aa, synthetic construct
  REGION 1..21: note = cycCMAP(1-21)[Leu6],Phe2,5,12/Trp
  SITE 6: note = MISC_FEATURE - is Fmoc-L-bishomopropargylglycine
  SITE 21: note = MISC_FEATURE - azido resin attached
RWGRWXRKIRRWRPKVTITIQ

SEQ ID NO: 8 - protein, 21 aa, synthetic construct
  REGION 1..21: note = cycCMAP(1-21)[Leu6],Phe5,12/Trp
  SITE 6: note = MISC_FEATURE - is Fmoc-L-bishomopropargylglycine
  SITE 21: note = MISC_FEATURE - azido resin attached
RFGRWXRKIRRWRPKVTITIQ

SEQ ID NO: 9 - protein, 21 aa, synthetic construct
  REGION 1..21: note = cycCMAP(1-21)[Leu6],Phe12/Trp
  SITE 6: note = MISC_FEATURE - is Fmoc-L-bishomopropargylglycine
  SITE 21: note = MISC_FEATURE - azido resin attached
RFGRFXRKIRRWRPKVTITIQ

SEQ ID NO: 10 - protein, 27 aa, synthetic construct
  REGION 1..27: note = Synthetic Peptide
GFRASGQITITVKPRFRRIKRLFRGFR

SEQ ID NO: 11 - protein, 21 aa, synthetic construct
  REGION 1..21: note = Synthetic Peptide
QITITVKPRFRRIKRLFRGFR

SEQ ID NO: 12 - protein, 18 aa, synthetic construct
  REGION 1..18: note = Synthetic Peptide
QITITVKPRFRRIKRLFR

SEQ ID NO: 13 - protein, 15 aa, synthetic construct
  REGION 1..15: note = Synthetic Peptide
QITITVKPRFRRIKR

SEQ ID NO: 14 - protein, 15 aa, synthetic construct
  REGION 1..15: note = Synthetic Peptide
QITITVKPRWRRIKR

SEQ ID NO: 15 - protein, 15 aa, synthetic construct
  REGION 1..15: note = Synthetic Peptide
QITITVKPRYRRIKR

SEQ ID NO: 16 - protein, 12 aa, synthetic construct
  REGION 1..12: note = Synthetic Peptide
QITITVKPRWRR

SEQ ID NO: 17 - protein, 26 aa, synthetic construct
  REGION 1..26: note = Synthetic Peptide
RFGRFLRKIRRFRPKVTITIQGSARF

SEQ ID NO: 18 - protein, 21 aa, synthetic construct
  REGION 1..21: note = Synthetic Peptide
RFGRFLRKIRRFRPKVTITIQ

SEQ ID NO: 19 - protein, 18 aa, synthetic construct
  REGION 1..18: note = Synthetic Peptide
RFLRKIRRFRPKVTITIQ

SEQ ID NO: 20 - protein, 15 aa, synthetic construct
  REGION 1..15: note = Synthetic Peptide
RKIRRFRPKVTITIQ

SEQ ID NO: 21 - protein, 15 aa, synthetic construct
  REGION 1..15: note = Synthetic Peptide
RKIRRWRPKVTITIQ

SEQ ID NO: 22 - protein, 15 aa, synthetic construct
  REGION 1..15: note = Synthetic Peptide
RKIRRYRPKVTITIQ

SEQ ID NO: 23 - protein, 12 aa, synthetic construct
  REGION 1..12: note = Synthetic Peptide
RRWRPKVTITIQ

SEQ ID NO: 24 - protein, 15 aa, synthetic construct
  REGION 1..15: note = Synthetic Peptide
RFGRFLRKIRRFRPK

SEQ ID NO: 25 - protein, 21 aa, synthetic construct
  REGION 1..21: note = Synthetic Peptide
RCGRFLRKIRPFRRKVTITRQ

SEQ ID NO: 26 - protein, 21 aa, synthetic construct
  REGION 1..21: note = Synthetic Peptide
RCGRFLRKIRPFRGKVTITRQ

SEQ ID NO: 27 - protein, 21 aa, synthetic construct
  REGION 1..21: note = Synthetic Peptide
RFGRFLRKIRRFRGKVTITRQ

SEQ ID NO: 28 - protein, 21 aa, synthetic construct
  REGION 1..21: note = Synthetic Peptide
RWGRWLRKIRRWRPKVTITRQ

SEQ ID NO: 29 - protein, 21 aa, synthetic construct
  REGION 1..21: note = Synthetic Peptide
RWGRWLRKIRRWRPKVTITIQ

SEQ ID NO: 30 - protein, 18 aa, synthetic construct
  REGION 1..18: note = Synthetic Peptide
RFLRKIRRFRPKVTITRQ

SEQ ID NO: 31 - protein, 18 aa, synthetic construct
  REGION 1..18: note = Synthetic Peptide
RFLRKIRRFRGKVTITRQ

SEQ ID NO: 32 - protein, 18 aa, synthetic construct
  REGION 1..18: note = Synthetic Peptide
RWLRKIRRWRPKVTITIQ

SEQ ID NO: 33 - protein, 18 aa, synthetic construct
  REGION 1..18: note = Synthetic Peptide
RWLRKIRRWRPKVTITRQ

SEQ ID NO: 34 - protein, 18 aa, synthetic construct
  REGION 1..18: note = Synthetic Peptide
RWLRKIRRWRGKVTITRQ

SEQ ID NO: 35 - protein, 21 aa, synthetic construct
  REGION 1..21: note = Synthetic Peptide
RFGRFLRKIRRFRPKVTITIQ

SEQ ID NO: 36 - protein, 21 aa, synthetic construct
  REGION 1..21: note = Synthetic Peptide
RCGRFLRKIRPFRRKVTITCQ

SEQ ID NO: 37 - protein, 21 aa, synthetic construct
  REGION 1..21: note = Synthetic Peptide
RFGRWLRKIRRYRGKVTITIQ